(19) United States
(12) Patent Application Publication
(10) Pub. No.: US 2007/0269469 A1
(43) Pub. Date: Nov. 22, 2007

(54) Hand-Held Depilatory and Exfoliating Device

(76) Inventor: Elizabeth Ann Hall-Maxwell, Loxahatchee, FL (US)

Correspondence Address: AKERMAN SENTERFITT, P.O. BOX 3188, WEST PALM BEACH, FL, 33402-3188, US

(21) Appl. No.: 11/751,324

(22) Filed: May 21, 2007

Related U.S. Application Data
(60) Provisional application No. 60/747,761, filed on May 19, 2006.

(51) Int. Cl. A61K 8/02

(52) U.S. Cl. 424/401

(57) ABSTRACT

A depilatory and skin exfoliating device comprising a central pad, an abrasive layer attached to one side of the pad, and a cleansing layer attached to the other side of the pad. By including both a depilatory device and a skin exfoliator, surface detritus and residue can be removed from the skin and the skin can be smoothed after the hair abrading process.

### CROSS-REFERENCE TO RELATED APPLICATIONS

The present application claims priority from U.S. Patent Provisional Application No. 60/747,761, filed May 19, 2006, the contents of which are herein incorporated by reference in their entirety.

### BACKGROUND OF THE INVENTION

The cosmetic business is a billion dollar industry, with many millions of dollars spent on hair removal systems. However, many of these systems can be dangerous to young or elderly users and are not always convenient to use. Additionally, many of the current hair removal processes can be quite painful, regardless of a users age. For example, blade shaving systems require the user to change and use at least one, if not four or sometimes even five razor blades. Electrical razors require a power source, waxing requires a significant amount of time, heat and pain, and plucking just isn't practical for hair found on the legs. Depilatory creams are usually messy and somewhat expensive, and have a disturbing smell because of their chemical composition. There have been, however, a few abrading systems developed and this invention improves upon the products in this field. No other previous invention has included both a depilatory device and a skin exfoliator for removing the surface detritus and residue and soothing the skin after the hair abrading process. Furthermore, no previously invented device can conform to the users grip and the contours of their skin.

### SUMMARY OF THE INVENTION

The present invention relates to a hand-held, contoured, manually manipulated hair removal depilatory and exfoliator device used to remove hair from the human body, in particular to a persons' legs, and to also soothe and remove surface detritus from the skin after completing the hair removal process. This device has at least one abrasive surface and one cleansing surface preferably made of a natural rubber material. The device removes hair from the body by “sanding” or rubbing the abrasive surface against the skin in a circular or orbital sweeping motion where hair removal is desired. The other side, composed of a natural rubber like material, is used to attract and remove the skin flakes, hair residue and surface detritus resulting from the abrasion. The result of using this device allows for safe, effective, and inexpensive temporary hair removal.

### BRIEF DESCRIPTION OF THE DRAWINGS

FIG. 1 is a perspective view of the exfoliator of the subject application;

FIG. 2 is a side elevational view of the exfoliator of FIG. 1; and

FIG. 3 is an end elevational view of the exfoliator of FIG. 1.

### DETAILED DESCRIPTION

The following embodiment is preferred, however, it is understood that the invention is not limited to the precise arrangements and instrumentalities shown.

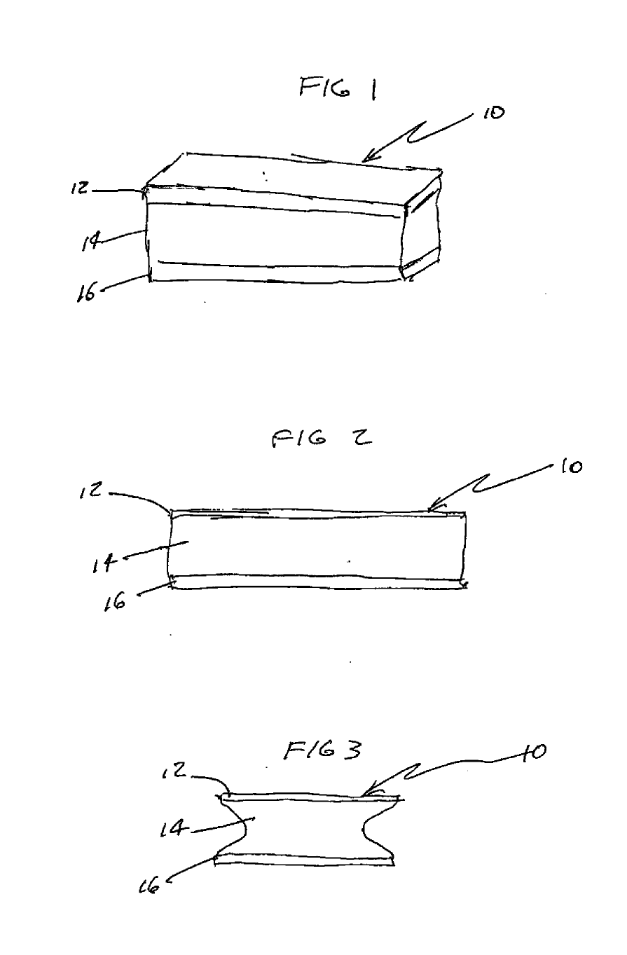

FIG. 1 illustrates a depilatory and skin exfoliating device 10 in accordance with the inventive arrangements. As shown in FIG. 1 the depilatory and exfoliating device 10 can include a porous pad 14 an abrasive layer 12 on top of the pad and a cleansing layer 16 preferably constructed of natural rubber like material affixed to the bottom side of the pad 14. FIG. 1 illustrates the abrasive layer 12 comprised of a Silicon Carbide Film attached via a lamination process to the top surface of the pad 14. In one arrangement, the porous pad 14 can be a Memory Foam sponge, however, the invention is not so limited as any other suitable conforming, pliable material may be used. An adhesive can be used to adhere the abrasive layer 12 to the pad 14, and an adhesive can be used to affix the abrasive particulates to the top layer of the pad 14. Alternatively, the abrasive layer 12 can also be attached via a proprietor process of using coated foam with an elastomeric polymer with added aggregate cured in a heat tunnel. In all arrangements, the abrasive layer 12 may be comprised of film, paper or foam coated with Aluminum Oxide or Silicon Carbide grains. Different levels of abrasiveness may be obtained by using different grains and sizes.

FIG. 2 illustrates a side view of the depilatory and exfoliating device 10. As shown in FIG. 2 the pad 14 or center portion of the device can be constructed of a Memory Foam center. In another arrangement, the pad 14 can be constructed of any other pliable material of which an abrasive layer 12 and a natural rubber like material layer 16 can be attached to the pad 12. It It can be made of any suitable material such as plastic, wood, rubber or neoprene. However, hard plastics and woods will not allow for deformation or customization of the unit.

The present invention provides a device 10 for removing hair from the body, via the abrasive coating 12 onto a block of foam 14, preferably Memory Foam®, and for removing dead skin cells and dander with a natural rubber material 16 adhered to the opposite side. In the preferred embodiment, the silicon carbide abrasive 12 or Aluminum Oxide grains are on one side of the foam 14 and the opposite side has a layer of a natural rubber-like material 16, such as Gonzo®, which is used to remove loose hair, dead skin cells and dander, and to apply lotions in order to soothe the skin after the hair removal process. The Gonzo® sponge 16 has an electrostatic charge and is able to pick up skin flakes and hair residue resulting from the abrasion. When used properly, this will not cause abrasions to the skin.

The abrasive layer 12 of this unit is attached via a lamination process with adhesive when coated film or paper is used as the abrasive, and is used to abrade or “sand” the top of the hair follicle off. Alternatively, there is also the potential to use a proprietor process of coated foam with an elastomeric polymer with added aggregate cured in a heat tunnel that provides the abrasive surface 12. The abrasive coating 12 or laminated coated film will use different grains to create different levels of abrasiveness. A 30 micron Silicon Carbide Film proves to be the most effective at this time. However, the abrasive surface 12 may be comprised of film, paper or foam coated with Aluminum Oxide or Silicon Carbide grains.

The opposite side of the sponge has a layer 16 of a natural rubber material, preferably Gonzo® material adhered to at least one of the remaining surfaces to attract and remove the skin flakes, hair residue and surface detritus resulting from the abrasion. The natural rubber material 16 attracts the dander and dead skin cells from the sanding process, and also gives the user a unit from which to apply lotions in order to soothe the skin.

The device 10 has a contoured shape that fits comfortably within a persons grip. This tapering shape (see FIG. 3) aides in a comfort experienced by the user when manipulating the device 10. The middle “sponge” portion 14, is made of a Memory Foam material or any other suitable material, preferably one that is pliable. The advantage to using a Memory Foam center in particular, and a contoured shape allows the user to have sustained gripping times and manipulation at a comfortable, custom level. Since the unit 10 is flexible and made of Memory Foam it will eventually conform to the configuration of the users hand grip and leg, in essence “remembering” the users unique grasp, making for a more comfortable and custom experience upon each use. Therefore, causing little, if any, fatigue or pain during the hair removal process. However, the device 10 may also be made of a plastic material, or any other suitable material where deformation or grip customization is not desirable or necessary.

After use, the unit 10 is simply wiped clean and left to dry, ready for use again within minutes. Also when used, the product is most effective on dry skin as opposed to wet.

1. A depilatory and skin exfoliating device comprising:
a central pad;
an abrasive layer attached to one side of the pad; and
a cleansing layer attached to the other side of the pad.

2. The device of claim 1 wherein the pad is porous.

3. The device of claim 2 wherein the porous pad is constructed of memory foam.

4. The device of claim 1 wherein the abrasive layer is comprised of a 30 micron Silicon Carbide Film.

5. The device of claim 4 wherein the Carbide Film is attached to the pad by a lamination process.

6. The device of claim 1 where the abrasive layer can be attached via a proprietary process of using coated foam with an elastomeric polymer with added aggregate cured in a heat tunnel.

7. The device of claim 1 where the abrasive layer can be comprised of film, paper, or foam coated with Aluminum Oxide or Silicon Carbide grains/particles.

8. The device of claim 1 wherein the cleansing layer is a natural rubber-like material.

9. The device of claim 8 wherein the rubber-like material is Gonzo®.

10. The device is claim 1 wherein the central pad is shaped like an hourglass.

11. A method of using a depilatory and skin exfoliating device comprising the steps of:
rubbing an abrasive layer against skin;
flipping the device over; and
rubbing a cleansing pad against the skin.
